(19) United States
(12) Patent Application Publication
(10) Pub. No.: US 2010/0168451 A1
(43) Pub. Date: Jul. 1, 2010

(54) PREPARATIVE METHOD OF DIHYDROCUCURBITACIN F-25-O-ACETATE AND THE USE THEREOF IN THE MANUFACTURE OF MEDICAMENTS FOR TREATING CANCERS

(76) Inventors: Xiane Fan, Yunnan (CN); Renwei Zhang, Yunnan (CN); Huijia Cheng, Yunnan (CN)

Correspondence Address: BYIP, LTD., P.O. BOX 1484, GENERAL POST OFFICE, HONG KONG, HK

(21) Appl. No.: 12/601,303

(22) PCT Filed: Oct. 19, 2007
(86) PCT No.: PCT/CN07/02874

§ 371 Date: Nov. 23, 2009

(51) Int. Cl. C07J 9/00

(52) U.S. Cl. 552/541

(57) ABSTRACT

A method for producing dihydrocucurbitacin F-25-0-acetate consisting of the steps as followed: percolating radix hemsleyae using acetone as solvents to obtain extract; eluting the extract in silica gel column using chloroform: methanol as eluting agent (gradient) to obtain the crude; recrystallizating the crude using methanol or ethanol to obtain di-hydrocucurbitacin F-25-O-acetate (purity:>98%). And the use of dihydrocucurbitacin F-25-O-acetate in the manufacture of medicaments for treating cancers of liver, lung, stomach, larynx, prostate and leukemia.

### FIELD OF THE INVENTION

The present invention relates to preparation method for dihydrocucurbitacin F-25-O-acetate (Cucurbitacin IIa) as a medical component, and the use thereof in the manufacture of medicaments for treating cancers.

### BACKGROUND OF THE INVENTION

Curcurbitacin is a mixture composed of curcurbitacin IIa and curcurbitacin IIb, and is isolated from the rhizomes of Hemsleya amalils Diels plant by Weixin Chen, et al (Chemical Journal, 1975, 33(1), 5). The curcurbitacin IIa is identified as 19-nor-9β-methyl-10à-3à-hydrogen-2β, 3à, 16à, 20, 25-5-hydroxy Δ5lanostene-11, 20-diketone-25-acetate (IIa), that is, a 23,24-cc dihydro-25-acetate of cucurbitacin F (Ic); the curcurbitacin IIb is identified as a 23,24-dihydro compound of cucurbitacin F (Ic), both of which are new compound, and used to prepare a curcurbitacin troche which is recorded in the pharmaceutical standard of YUNAN province in the year of 1974 for treating enteritis of bacillary dysentery, tracheitis and acute tonsillitis.

In order to find plant resource for extracting curcurbitacin, some scholars did resource chemical study to plant of present genus, and isolated curcurbitacin IIa and curcurbitacin IIb from Hemsleya maerosperma (c.y.wu), H, dolichocarp (W. J. cheng) and H, emiensil (L. T. Sent). Same extraction method as the chen's was employed in such studies, that is, ethanol is used to extract crude drug, then the extract is extracted by ethyl acetate in water, then the extract is subjected to the silica gel column, then eluted by a mixed solvent comprising chloroform, acetone and ethyl acetate in different ratio, then crude product is obtained by recycling the eluant, and curcurbitacin IIa and curcurbitacin IIb may be obtained by using ethanol to recrystallizate the crude product (Reference Document: Rui-Lin-Nie et al. planta Medica 1984:322; Yaqing Si et al, YunNan Propagation Study, 1990, 12(4), 460; Chinese Herbal Medicine 1995, 26 (12), 619).

A China patent application CN 01102620.0 discloses “A Nano-curcurbitacin medicament preparation and Its Preparation Method”, which takes nano-curcurbitacin as raw material, and prepares according to certain proportion, and then the resultant new medicament has a particle fineness range from 1200-1500 mesh, and a particle size range from 0.1-200 nm, most of which has a particle size less than 100 nm, and has a new physical property. In such application, technical steps of microwave extraction, reduced pressure condense and supersonic jet are employed. A China patent application CN 03159197.3 discloses “A Curcurbitacin Dripping Pill and Its Preparation Method”, wherein, one part by weight of curcurbitacin power which is superfine comminuted is added into one to ten parts by weight of fusion base, and then mixed uniformly. After that dropping method is used for condensing pills in refrigerant, and then the refrigerant is removed for such curcurbitacin dripping pill. A China patent application CN 02134164.8 discloses “A curcurbitacin derivative, Its Preparation Method and Its use as an antimicrobial”, which provide a curcurbitacin derivative which is different from the prior art, and its preparation method, and an antimicrobial containing such compound as effective content.

At present, no report about use of curcurbitacin IIa in anti-cancer medicament has been found.

### SUMMARY OF THE INVENTION

An object of this invention is to provide a preparation method of curcurbitacin IIa.

Another object of this invention is to provide use of curcurbitacin IIa in preparing anti-cancer medicament.

According to one aspect of the present invention, a preparation method of curcurbitacin IIa is provided, comprising the following steps:

a, the tuber of Hemsleya amalils Diels plant is obtained and comminuted, then extracted by a percolation method, using acetone of 18-26 weight times as solvent, then the extractant is recycled in water bathe of 60-70° C. for extract paste;

b. the obtained extract paste is mixed with silica gel of 1-2 weight times, then dried and subjected to the silica gel column, then gradient eluted by a mixed solvent comprising chloroform and methanol, wherein a volume concentration of the methanol is 2-10%, then the eluent is recycled and crude product is obtained;

c. the crude product is heated and dissolved in a water bath using methanol or ethanol, then the obtained solution is filtered and placed in a room temperature for recrystallization, then the obtained prismy crystals is the curcurbitacin IIa of 98% purity.

The Hemsleya amalils Diels plant in the step a is Hemsleya maerosperma, Hemsleya dolichocarpa W. J. Chang, and Hemsleya emeiensis L. T. Shen and so on. In the step b, at first pure chloroform is used to elute impurity, and then a mixed solvent comprising chloroform and methanol is used for gradient eluting. The curcurbitacin IIa obtained in step c, is identified to have a purity of 98% and a melting point of 226-228° C. by HPLC, and after an analyse of ultraviolet spectroscopic, IR spectroscopic, nuclear magnetic resonance and mass spectrum, the curcurbitacin IIa is determined, and its structural formula is disclosed as follows:

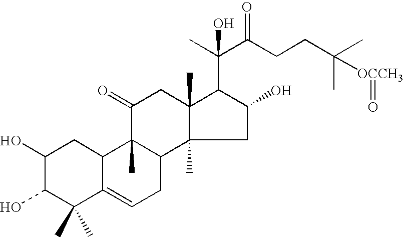

The present invention relates to use of curcurbitacin IIa in preparing medicament for treat hepatic cancer.

The present invention relates to use of curcurbitacin IIa in preparing medicament for treat lung cancer.

The present invention relates to use of curcurbitacin IIa in preparing medicament for treat gastric cancer.

The present invention relates to use of curcurbitacin IIa in preparing medicament for treat laryngeal cancer.

The present invention relates to use of curcurbitacin IIa in preparing medicament for treat prostate cancer.

The present invention relates to use of curcurbitacin IIa in preparing medicament for treat leukemia.

Pharmacodynamics Test Information:

The curcurbitacin IIa monomer is determined by pharmacological anti-cancer test in vitro and in vivo to have a function in suppression or kill cancer cell in the people's body. The Ic 50 (μg/ml) for growth of the human lung cancer cells is 1.5, which is almost the same as the cisplatin, which is 1.6.

The anti-cancer experiments on mouse in vivo depends on the type of the preparation and the way of medicament administration. Comparing with oral administration, injection administration has a better effect, and the emulsion intravenous injection has a lung cancer inhibition efficiency up to 59%.

Anti-Cancer Experiments:

1. Screening research in vitro, employing modified MTT of anticancer substance activity.

Cancer Cell Strain:

| | |
|---|---|
| HL60 | human leukemia cell strain |
| A549 | human lung cancer cell strain |
| SGC-7901 | human gastric cancer cell strain |
| Bel-7402 | human hepatic cancer cell strain |
| SMMC-7721 | human hepatic cancer cell strain |
| HEP-2 | human laryngeal cancer cell strain |
| GLC-15 | human lung cancer cell strain |
| PC-3 | human prostate cancer cell strain |

Results of the Research:

Ic 50 (μg/ml) for the growth of seven strains of tumour cells

| | Cell Strain | | | | | | | |
|---|---|---|---|---|---|---|---|---|
| Test Medicament | HL60 | A549 | SGC-7901 | Bel-7402 | SMMC-7721 | HEP-2 | GLC-15 | PC-3 |
| Curcurbitacin | 43.22 | 1.5 | 8.71 | >100 | 22.26 | 15.82 | 14.57 | 10.05 |
| IIa | | | | | | | | |
| DDP(Cisplatin) | 0.695 | 1.6 | 0.89 | 2.31 | 0.21 | 0.3 | 1.72 | 1.85 |

It can be seen from the above table that, in the present experiment condition, the Curcurbitacin IIa shows significant kill function to the human body tumour cells, however, it also shows significant difference between tumour cells from different tissue source, which means the Curcurbitacin IIa shows relative selectivity to different tissue cell.

2. Research in Vivo

After inoculating Kunming murine for H22 hepatic cancer and murine Lewis lung cancer, three kinds of preparation, i.e., suspension, emulsion, and lyophilized preparation, are administrated in three different ways of administration comprising gastric perfusion, intraperitoneal injection and intravenous injection, and the administration dose is high, middle and low respectively. Then the curative effect is compared with positive control group and the tumour inhibition efficiency is calculated, and the results are shown as follows:

TABLE I
The curative effect research for Curcurbitacin IIa suspension on mouse H22 liver
cancer through gastric perfusion

| | | | | Animal | | |
|---|---|---|---|---|---|---|
| | | | | body | | |
| | | Administration | Animal | weight | Tumour | |
| | dosage | method | number | (g) | weight (g) | Inhibition |
| sample | mg/kg/d | igx10qd | Start/end | Start/end | X ± SD | efficiency |
| Curcurbitacin | 90 | igx10qd | 10/10 | 19.1/25.7 | 1.96 ± 0.16 | 38.17 |
| IIa | | | | | | |
| Curcurbitacin | 60 | igx10qd | 10/10 | 19.3/26.3 | 2.14 ± 0.20 | 32.09 |
| IIa | | | | | | |
| Curcurbitacin | 30 | igx10qd | 10/10 | 19.0/26.0 | 2.31 ± 0.21 | 27.13 |
| IIa | | | | | | |
| Positive | 30 | igx7qd | 10/10 | 19.4/23.1 | 0.33 ± 0.013 | 89.59 |
| control | | | | | | |
| CTX | | | | | | |
| Negative | Corresponding | igx10qd | 10/10 | 19.3/26.5 | 3.17 ± 0.32 | |
| control | solvent | | | | | |

Compared with the negative control, p < 0.01. This research has significant difference.

TABLE II
The curative effect research for Curcurbitacin IIa suspension on mouse H22
liver cancer through intraperitoneal administration

| | | | | Animal | | |
|---|---|---|---|---|---|---|
| | | | | body | | |
| | | Administration | Animal | weight | Tumour | |
| | dosage | method | number | (g) | weight (g) | Inhibition |
| sample | mg/kg/d | igx10qd | Start/end | Start/end | X ± SD | efficiency |
| Curcurbitacin | 90 | igx10qd | 10/9 | 21.1/27.6 | 1.31 ± 0.12 | 57.05 |
| IIa | | | | | | |
| Curcurbitacin | 60 | igx10qd | 10/10 | 21.0/24.6 | 1.49 ± 0.12 | 51.05 |
| IIa | | | | | | |
| Curcurbitacin | 30 | igx10qd | 10/10 | 20.8/25.4 | 1.95 ± 0.12 | 36.07 |
| IIa | | | | | | |
| Positive | 30 | igx7qd | 10/10 | 21.2/23.1 | 0.33 ± 0.13 | 89.18 |
| control | | | | | | |
| CTX | | | | | | |
| Negative | Corresponding | igx10qd | 20/20 | 20.8/25.9 | 3.05 ± 0.28 | |
| control | solvent | | | | | |

Compared with the negative control, p < 0.01.

TABLE III
The curative effect research for Curcurbitacin IIa emulsion on mouse H22 liver
cancer through intravenous administration

| | | | | Animal | | |
|---|---|---|---|---|---|---|
| | | | | body | | |
| | | Administration | Animal | weight | Tumour | |
| | dosage | method | number | (g) | weight (g) | Inhibition |
| sample | mg/kg/d | ivx10qd | Start/end | Start/end | X ± SD | efficiency |
| Curcurbitacin | 15 | ivx10qd | 10/10 | 19.5/25.3 | 1.48 ± 0.11 | 55.02 |
| IIa | | | | | | |
| Curcurbitacin | 10 | ivx10qd | 10/10 | 19.3/25.9 | 1.76 ± 0.08 | 44.12 |
| IIa | | | | | | |
| Curcurbitacin | 5 | ivx10qd | 10/10 | 19.4/25.1 | 2.13 ± 0.20 | 32.38 |
| IIa | | | | | | |
| Positive | 30 | ivx7qd | 10/10 | 19.6/22.9 | 0.31 ± 0.13 | 90.16 |
| control | | | | | | |
| CTX | | | | | | |
| Negative | Corresponding | ivx10qd | 10/10 | 19.4/26.0 | 3.15 ± 0.44 | |
| control | solvent | | | | | |

Compared with the negative control, p < 0.01.

TABLE IV
The curative effect research for Curcurbitacin IIa emulsion on mouse Lewis
lung cancer through intravenous administration

| | | | | Animal | | |
|---|---|---|---|---|---|---|
| | | | | body | | |
| | | Administration | Animal | weight | Tumour | |
| | dosage | method | number | (g) | weight (g) | Inhibition |
| sample | mg/kg/d | ivx10qd | Start/end | Start/end | X ± SD | efficiency |
| Curcurbitacin | 15 | ivx10qd | 10/10 | 19.5/22.1 | 1.16 ± 0.16 | 59.30 |
| IIa | | | | | | |
| Curcurbitacin | 10 | ivx10qd | 10/10 | 19.1/22.9 | 1.50 ± 0.14 | 47.37 |
| IIa | | | | | | |
| Curcurbitacin | 5 | ivx10qd | 10/10 | 19.3/22.4 | 1.69 ± 0.11 | 40.76 |
| IIa | | | | | | |
| Positive | 30 | ivx10qd | 10/10 | 18.7/18.1 | 0.362 ± 0.03 | 87.76 |
| control | | | | | | |
| CTX | | | | | | |
| Negative | Corresponding | ivx10qd | 20/20 | 18.9/23.0 | 2.85 ± 0.23 | |
| control | solvent | | | | | |

Compared with the negative control, p < 0.01.

TABLE V
The curative effect research for Curcurbitacin IIa lyophilized preparation
on mouse H22 liver cancer through intravenous administration

| | | | | Animal | | |
|---|---|---|---|---|---|---|
| | | Administration | Animal | body | Tumour | |
| | dosage | method | number | weight (g) | weight (g) | Inhibition |
| sample | mg/kg/d | ivx10qd | Start/end | Start/end | X ± SD | efficiency |
| Curcurbitacin | 15 | ivx10qd | 10/10 | 20.4/26.7 | 1.61 ± 0.18 | 46.15 |
| IIa | | | | | | |
| Curcurbitacin | 10 | ivx10qd | 10/10 | 20.7/25.9 | 1.82 ± 0.19 | 39.13 |
| IIa | | | | | | |
| Curcurbitacin | 5 | ivx10qd | 10/10 | 20.5/26.8 | 2.06 ± 0.18 | 31.1 |
| IIa | | | | | | |
| Positive | 30 | Ipx7qd | 10/10 | 20.9/24.1 | 0.402 ± 0.14 | 86.56 |
| control | | | | | | |
| CTX | | | | | | |
| Negative | Corresponding | ivx10qd | 20/20 | 20.03/27.3 | 2.99 ± 0.30 | |
| control | solvent | | | | | |

TABLE VI
The curative effect research for Curcurbitacin IIa lyophilized preparation on
mouse Lewis lung cancer through hypodermic inoculation

| | | | | Animal | | |
|---|---|---|---|---|---|---|
| | | | | body | | |
| | | Administration | Animal | weight | Tumour | |
| | dosage | method | number | (g) | weight (g) | Inhibition |
| sample | mg/kg/d | ivx10qd | Start/end | Start/end | X ± SD | efficiency |
| Curcurbitacin | 15 | ivx10qd | 10/10 | 18.0/20.7 | 1.20 ± 0.20 | 51.20 |
| IIa | | | | | | |
| Curcurbitacin | 10 | ivx10qd | 10/10 | 17.7/21.1 | 1.34 ± 0.18 | 45.31 |
| IIa | | | | | | |
| Curcurbitacin | 5 | ivx10qd | 10/10 | 18.1/20.5 | 1.56 ± 0.14 | 36.33 |
| IIa | | | | | | |
| DDP | 7 | Ipx2, the first, | 10/10 | 18.4/18.1 | 0.172 ± 0.06 | 92.98 |
| | | third days | | | | |
| Negative | NS | ivx10qd | 20/20 | 17.9/22.4 | 2.45 ± 0.22 | |
| control | | | | | | |

Compared with the negative control, p < 0.01.

### CONCLUSION

The above experiments prove that, the Curcurbitacin IIa has significant inhibition or kill efficiency to various cancers including hepatic cancer, lung cancer, gastric cancer, laryngeal cancer, prostate cancer and leukemia etc, and its effect depends on the type of the preparation, the way of medicament administration, and the administration dose.

### BRIEF DESCRIPTION OF THE DRAWINGS

FIG. 1 is a high performance liquid phase chromatogram.

### DETAILED DESCRIPTION OF THE PREFERRED EMBODIMENT

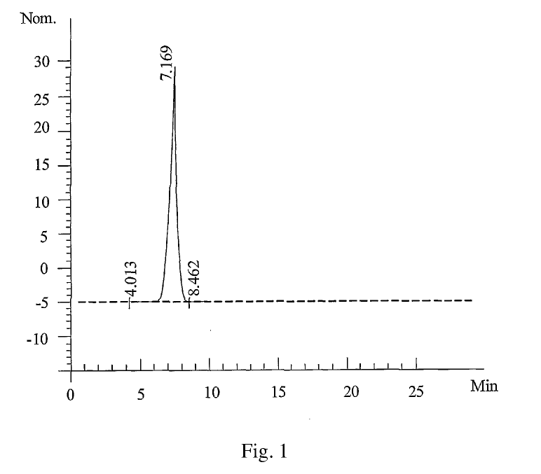

Embodiment 1: 5 kg Hemsleya maerosperma in the area of Qiaojia County of Yunnan Province is comminuted into middle power, and put into a seepage container, and then marinated for 48 hours at 15-25° C. after adding acetone of 24 weight times. Then a percolation method according to Chinese Pharmacopoeia is employed for percolating. After that, the extractant is recycled, then mixed with silica gel of equal weight, dried at 50-70° C., and subjected to the silica gel column, then gradient eluted by a mixed solvent comprising chloroform and methanol. Then an eluent with 3-5% concentrate of methanol is collected, and is recycled and then crude product is obtained. Methanol is employed to crystallize for several times, then prismy crystal with a melting point of 226-228° C. is obtained, the purity of which is identified to be 98% by HPLC. Referring to FIG. 1, after analyse of ultraviolet spectrum, IR spectrum, nuclear magnetic resonance and mass spectrum, the prismy crystal is determined to be curcurbitacin IIa.

Using said curcurbitacin IIa as a monomer component, medicament in various kinds of form, such as suspension, emulsion, lyophilized preparation, nano-lyophilized preparation, troche, pill, dropping pill, capsule, release control agent, microcapsule, lipid agent and so on, can be prepared, used to treat hepatic cancer, lung cancer, gastric cancer, laryngeal cancer, prostate cancer and leukemia.

What is claimed is:

1. A preparation method of curcurbitacin IIa, comprising the following steps:
a, the tuber of Hemsleya amalils Diels plant is obtained and comminuted, then extracted by a percolation method, using acetone of 18-26 weight times as solvent, then the extractant is recycled in water bathe of 60-70° C. for extract paste;
b. the obtained extract paste is mixed with silica gel of 1-2 weight times, then dried and subjected to the silica gel column, then gradient eluted by a mixed solvent comprising chloroform and methanol, wherein a volume concentration of the methanol is 2-10%, then the eluent is recycled and crude product is obtained;
c. the crude product is heated and dissolved in a water bath using methanol or ethanol, then the obtained solution is filtered and placed in a room temperature for recrystallization, then the obtained prismy crystals is the curcurbitacin IIa of 98% purity.

2. The preparation method of curcurbitacin IIa according to claim 1, wherein the Hemsleya amalils Diels plant is Hemsleya maerosperma, Hemsleya dolichocarpa W. J. Chang, and Hemsleya emeiensis L. T. Shen.

3. The preparation method of curcurbitacin IIa according to claim 1, wherein in the step b, at first pure chloroform is used to elute impurity, and then a mixed solvent comprising chloroform and methanol is used for gradient eluting.

4. Use of curcurbitacin IIa according to claim 1 in preparing medicament for treat hepatic cancer.

5. Use of curcurbitacin IIa according to claim 1 in preparing medicament for treat lung cancer.

6. Use of curcurbitacin IIa according to claim 1 in preparing medicament for treat gastric cancer.

7. Use of curcurbitacin IIa according to claim 1 in preparing medicament for treat laryngeal cancer.

8. Use of curcurbitacin IIa according to claim 1 in preparing medicament for treat leukemia.

9. Use of curcurbitacin IIa according to claim 1 in preparing medicament for treat prostate cancer.
